(19) United States
(12) Plant Patent Application Publication
(10) Pub. No.: US 2014/0283243 P1
(43) Pub. Date: Sep. 18, 2014

(54) Variety of tibouchina named 'Peace Baby'

(50) ;

(71) Applicant: Terry Keogh, Victoria Point (AU)

(72) Inventor: Terry Keogh, Victoria Point (AU)

(21) Appl. No.: 13/815,677

(22) Filed: Mar. 14, 2013

(51) Int. Cl. A01H 5/00

(52) U.S. Cl. PLT/226

(57) ABSTRACT

‘Peace Baby’ is a new variety of Tibouchina plant that is suitable for planting in gardens, containers, and in landscaping having tolerance to low temperatures and bright red buds turning into white flowers.

### BOTANICAL CLASSIFICATION

Tibouchina×hybrida

### VARIETAL DENOMINATION

‘PEACE BABY’

### BACKGROUND OF THE NEW PLANT

The present invention comprises a new and distinct cultivar of Tibouchina plant, botanically known as Tibouchina×hybrida and hereinafter referred to by the cultivar name ‘Peace Baby’.

The new and ornamental variety was selected by the breeder as a result of an ongoing breeding program that has been conducted by the inventor for more than thirty years at the inventor's nursery in Victoria Point, Queensland, Australia. The purpose of the breeding program is to produce novel varieties of Tibouchina in varying plant sizes, flower colors, and which are well-suited to a range of climate zones. ‘Peace Baby’ is a hybrid plant derived from the deliberate controlled cross-pollination of the female parent, an individual plant of Tibouchina organensis, named ‘Totally Moonstruck’, and the male parent, an individual plant of Tibouchina mutabilis name ‘Jazzie’. The inventor emasculated flowers of the female parent and applied pollen that was freshly collected from the male parent in 2002. The parent plants were isolated to prevent open pollination. In 2005, the inventor selected ‘Peace Baby’ as a seedling that had been raised from the cross-pollination described herein. Selection was based on the criteria of habit, flower color, plant dimensions, and cold temperature tolerance. The plant was then propagated in 2007 via vegetative cuttings to produce a new generation for final evaluation.

Further, asexual reproductions of ‘Peace Baby’ at the same location has demonstrated that the combination of characteristics as herein disclosed for the new cultivar are retained and reproduced true to type through more than six successive generations of asexual reproduction.

The color references made herein are to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Peace Baby’ which, in combination, distinguish this Tibouchina plant as a new and distinct cultivar:

1. Compact size and with rounded plant growth (dwarf form);

2. Mid-sized white flowers;

3. Dense plant habit;

4. Bright red buds;

5. Moderate to fast vigor;

5. Low temperature tolerance; and

6. Woody, long-lived shrub.

The new variety differs from ‘Totally Moonstruck’ in that ‘Peace Baby’ has a more consistent flower color, smaller plant size at maturity, and better cold tolerance. ‘Peace Baby’ differs from its pollen parent in flower color and shorter plant dimensions at maturity.

### BRIEF DESCRIPTION OF THE DRAWINGS

The accompanying photographic drawings illustrate the new variety, with the colors being as nearly true as is possible with color illustrations of this type:

FIG. 1 is close-up photograph of the flowers of the new variety; and

FIG. 2 is a photograph of an entire plant of the new variety

### PROPAGATION

Time to initiate roots: About 14-18 days at approximately 21° C.
Time to produce a rooted cutting: About 25-30 days at 21° C.

### PLANT

Plant spread: Approximately 24 cm.
Plant height: 21 cm.
Growth rate: Moderate to fast.
Lateral branches:
   Length.—Approximately 17.5 cm.
   Diameter.—Approximately 0.3 cm.
   Quantity.—About 22.
Age of plant when described: Approximately 14 weeks from a rooted cutting.
Growth habit: Compact; rounded.
Cold tolerance: Medium to strong.
Height at maturity: 1 m.
Width at maturity: 1 m.
Stem:
Branching habit: Strong, basal branching.
Main stem diameter: 0.35 cm.
Lateral stem length (average): 7 cm.
Lateral stem width (average): 0.17 cm.
Internode length (range): 2.2 cm to 7.5 cm.
Shape: Square in cross section.
Surface: Rough.
Branch color:
   Juvenile.—146D.
   Semi-ripe.—146B.
   Mature.—199B.
Foliage:
Leaf:
   Arrangement.—Opposite.
   Division.—Simple.
   Margin.—Entire.
   Shape.—Ovate.
   Base.—Cuneate to obtuse.
   Apex.—Acute.
   Surface (adaxial).—Pubescent.
   Surface (abaxial).—Pubescent.
   Texture (adaxial surface).—Prominently ribbed.
   Texture (abaxial surface).—Prominently ribbed.
   Attachment.—Petiolate.
   Length (range excluding petiole).—3.5 cm to 9 cm.
   Width (range excluding petiole).—2 cm to 3.8 cm.
   Color (adaxial surface).—147A and at the margin.
   Color (abaxial surface).—146B.
   Color of margin of juvenile foliage not fully expanded.—184A.
Foliar fragrance: None observed to date.
Venation:
   Pattern.—Reticulate; 3 primary parallel veins.
   Color (adaxial surface).—147B.
   Color (abaxial surface).—146D.
   Prominence (adaxial surface).—Depressed.
   Vein prominence (abaxial surface).—Raised.
Petiole:
   Length (range).—1.3 cm to 2.2 cm.
   Color.—184A and 178B.
   Surface.—Pubescent.
   Shape.—Cylindrical, but flattened on the adaxial surface.
Flower:
   Shape.—Rotate.
   Aspect.—Facing upward and outward.
   Inflorescence type.—Solitary.
   Persistent or self-cleaning.—Self-cleaning.
   Blooming period.—Summer through fall; can bloom sporadically during other times.
   Inflorescence fragrance.—None observed to date.
Petals:
   Number.—5.
   Margin.—Entire.
   Apex.—Obtuse.
   Base.—Broadly attenuate.
   Shape.—Obovate.
   Surface (abaxial and adaxial).—Glabrous.
   Fused or unfused.—Unfused.
Buds:
   Shape.—Elliptic.
   Color.—181A.
   Surface.—Pubescent.
   Apex.—Acute.
Calyx:
   Shape.—Cupule.
   Color.—Ranges between 181A and 44A as bud develops and flower opens.
Sepals:
   Quantity.—5 in number.
   Surface (adaxial).—Smooth.
   Surface (abaxial).—Puberulent and hair color is 197D.
   Color (adaxial surface).—Ranges between 181A and 144D.
   Color (abaxial surface).—181A.
   Margin.—Entire.
   Apex.—Acute.
   Base.—Truncate.
   Fused or unfused.—Unfused.
Peduncle:
   Dimensions.—1 m in length and 2 m in diameter.
   Shape.—Cylindrical.
   Surface.—Pubescent.
Reproductive organs:
Stamens:
   Number.—10.
   Color.—84B.
   Length.—2 cm.
   Width.—1 mm.
Anthers:
   Color.—Ranges between N89A and 155A.
   Shape.—Sickle.
Pollen: None observed.
Pistil:
   Number.—1.
   Shape.—Columnar.
Stigma:
   Shape.—Flattened.
Ovary:
   Color.—N44A.
   Shape.—Cupule.
   Surface.—Pubescent.
   Position.—Inferior.
Seeds: No seeds have been observed to date.

I claim:

1. A new and distinct variety of Tibouchina plant named ‘Peace Baby’ as herein described and illustrated.
